(19) United States
(12) Patent Application Publication
(10) Pub. No.: US 2012/0091355 A1
(43) Pub. Date: Apr. 19, 2012

(54) SELECTIVE DETECTION OF AROMATIC ALPHA-AMINO ACIDS AND DERIVATIVES THEREOF

(76) Inventors: Chebrolu P. RAO, Mumbai (IN); Jugun Prakash Chinta, Mumbai (IN); Atanu Mitra, Mumbai (IN)

(21) Appl. No.: 12/907,590

(22) Filed: Oct. 19, 2010

(51) Int. Cl. G01N 21/59; G01N 21/64; C07H 5/04

(52) U.S. Cl. 250/372; 536/55; 250/459.1

(57) ABSTRACT

Provided here are complexes useful in the detection of aromatic alpha-amino acids and peptides incorporating aromatic alpha-amino acids, and methods for detecting aromatic alpha-amino acids and peptides incorporating aromatic alpha-amino acids. Accordingly, provided herein are complexes comprising a compound of Formula I:

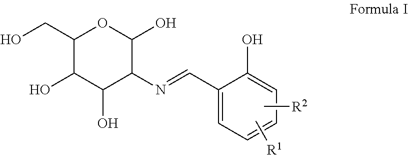

and an aromatic alpha-amino acid, a peptide incorporating an aromatic alpha-amino acid, or a salt or ester of any of the foregoing. Also, provided herein are methods for detecting an aromatic alpha-amino acid, a peptide incorporating an aromatic alpha-amino acid, or a salt or ester of any of the foregoing, by using the fluorescence intensity enhancement, or the hypochromic shift, of the compound of Formula I.

### BACKGROUND

It is necessary to detect aromatic alpha-amino acids for a variety of reasons, including, but not limited to determining protein structure and amount. Moreover, the detection and measurement of such amino acids in food, water and soil samples may provide a useful way of assessing their nutritive value. Detection of aromatic alpha-amino acids in biological samples may also be used to assess amino acid metabolism, including defective amino acid metabolism, or certain types of organ damage.

Aromatic alpha-amino acids may be detected by measuring their ultraviolet (UV) absorbance at 280 nm or 254 nm, or their fluorescence properties. Aromatic alpha-amino acids may also be detected using high performance liquid chromatography (HPLC), thin layer chromatography (TLC), and metal ion-based and macromolecule-based sensors employing emission and absorption changes. However, such methods may suffer from low sensitivity and/or selectivity. Thus, methods for selectively assessing low levels of aromatic alpha-amino acids in biological and other samples would be useful.

### SUMMARY

The present technology provides complexes useful in the detection of aromatic alpha-amino acids and peptides incorporating aromatic alpha-amino acids, and methods for detecting aromatic alpha-amino acids and peptides incorporating aromatic alpha-amino acids.

Accordingly, in one aspect, the present technology provides complexes including a compound of Formula I:

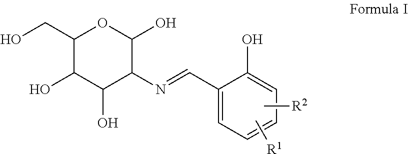

and an aromatic alpha-amino acid, a peptide incorporating an aromatic alpha-amino acid, or a salt or ester of any of the foregoing, wherein R¹ and R² are hydrogen or R¹ and R² together with the carbon atoms to which they are bonded form a phenyl ring.

In some embodiments, R¹ and R² are hydrogen. In other embodiments, the compound of Formula I is 1-(D-glucopyranosyl-2′-deoxy-2′-iminomethyl)-2-hydroxybenzene (or simply, compound L) of formula:

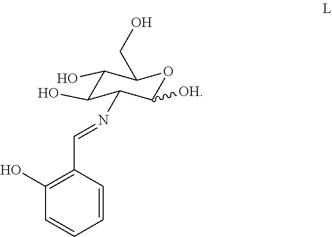

In some embodiments, the aromatic alpha-amino acid or the peptide incorporating an aromatic alpha-amino acid has the Formula II:

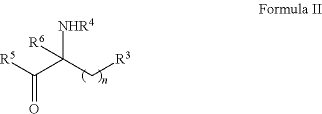

wherein

R³ is:

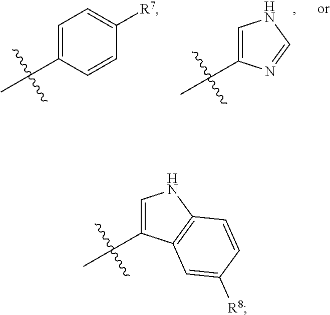

wherein

R⁷ is hydrogen or hydroxyl;

R⁸ is hydrogen or hydroxyl;

R⁴ is hydrogen or an amino acyl moiety wherein the amino acyl moiety is an acyl moiety derived from an alpha-amino acid or a peptide;

R⁵ is hydroxyl or an amino moiety derived from an alpha-amino acid or a peptide;

R⁶ is hydrogen or methyl; and

n is 0 or 1.

In some embodiments, R⁴ is hydrogen. In other embodiments, R⁵ is hydroxyl. In some embodiments, R⁴ is hydrogen and R⁵ is hydroxyl. In other embodiments, R⁶ is hydrogen, and/or, n is 1. In other embodiments, the aromatic alpha-amino acid is phenylalanine, tyrosine, histidine, or tryptophan.

In certain embodiments of the complex, the compound of Formula I is compound L. In some embodiments, the molar ratio of the compound of Formula I and the aromatic alpha-amino acid or the peptide, or a salt or ester thereof ranges from about 1:1 to about 1:2.

In another aspect, the present technology provides a method of testing for the presence (or absence) of an aromatic alpha-amino acid, a peptide incorporating an aromatic alpha-amino acid, or a salt or ester of any of the foregoing including: detecting the fluorescence emission intensity of a test sample including a compound of Formula I as shown above, and comparing the detected fluorescence emission intensity of the test sample to that of a control sample, wherein a change in the fluorescence emission intensity of the test sample relative to the control sample indicates the presence of the aromatic alpha-amino acid the peptide incorporating an aromatic alpha-amino acid, or the salt or ester of any of the foregoing. In some embodiments, the change in the fluorescence emission intensity of the test sample is an increase, and in other embodiments, the change is a decrease. In certain embodiments of the methods, an unchanged fluorescence emission intensity of the test sample relative to the control sample indicates the absence of the aromatic alpha-amino acid, the peptide incorporating an aromatic alpha-amino acid, or the salt or ester of any of the foregoing in the test sample.

In some embodiments of the methods, for the compound of Formula I, R¹ and R² are hydrogen. In other embodiments, the compound of Formula I is compound L.

In some embodiments, the aromatic alpha-amino acid or the peptide incorporating an aromatic alpha-amino acid has the Formula II as shown above. In some embodiments, for the compound of Formula II, R⁴ is hydrogen. In other embodiments, R⁵ is hydroxyl. In still other embodiments, R⁴ is hydrogen and R⁵ is hydroxyl. In some embodiments, R⁶ is hydrogen and/or n is 1.

In other embodiments of the methods, the aromatic alpha-amino acid is phenylalanine, tyrosine, histidine, tryptophan or a mixture of any two or more thereof. In some such embodiments, the compound of Formula I is compound L.

In some embodiments, the fluorescence emission intensity is detected in the range from about 330 nm to about 500 nm.

In some embodiments, the test sample is an aqueous, biological, food or environmental sample. In some embodiments, the control sample contains substantially the same amount of the compound of Formula I as the test sample but lacks the aromatic alpha-amino acid, the peptide incorporating an aromatic alpha-amino acid, or the salt or ester thereof.

In another aspect, the present technology provides a method of testing for the presence (or absence) of tryptophan or a salt or ester thereof including:
detecting the ultraviolet absorption intensity of a test sample including a compound of Formula I, and
comparing the detected ultraviolet absorption intensity of the test sample to that of a control sample,
wherein a change (e.g., a decrease) in the ultraviolet absorption intensity (or a hypochromic shift) of the test sample relative to the control sample indicates the presence of tryptophan or the salt or ester thereof in the test sample. In some embodiments, an unchanged ultraviolet absorption intensity of the test sample relative to the control sample indicates the absence of tryptophan or the salt or ester thereof in the test sample.

In some embodiments of the methods, the compound of Formula I is compound L. In certain embodiments, the ultraviolet absorption intensity is detected at about 214 nm. In some embodiments, the test sample is an aqueous, biological, food or environmental sample. In other embodiments, the control sample contains substantially the same amount of the compound of Formula I as the test sample but lacks tryptophan or a salt or ester thereof.

The foregoing summary is illustrative only and is not intended to be limiting in any way. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1(a) depicts an illustrative embodiment of the plots of relative fluorescence emission intensity (I/I₀) vs. [A.A]/[L] mole ratio in which I is measured fluorescence emission intensity; I₀ is initial fluorescence emission intensity; [A.A] is the concentration of amino acid in moles/liter; and [L] is the concentration of 1-(D-glucopyranosyl-2′-deoxy-2′-iminomethyl)-2-hydroxybenzene in moles/liter. The symbols are: ▪=His, =Phe, ▴=Trp, and ▾=Tyr. FIG. 1(b) depicts an illustrative embodiment of a bar diagram indicating number of times of fluorescence emission intensity enhancement observed with all the twenty amino acids.

FIG. 2 depicts an illustrative embodiment of the plots of concentration versus fluorescence emission intensity for certain aromatic alpha-amino acids measured while maintaining a 1:1 mole ratio for [amino acid]/[L]. The symbols are: ▪=GluSI (glucosyl salicylimine, the receptor alone), =His, ▴=Phe, ▾=Trp, and [symbol] =Tyr.

FIG. 3(a) depicts an illustrative embodiment of histograms showing the fluorescence emission intensity enhancement of compound L when titrated against amino acids (shaded) and their corresponding methyl esters (unshaded). FIG. 3(b) depicts an illustrative embodiment of histograms showing the fluorescence emission intensity enhancement of compound L when titrated against the aromatic carboxylic acid, benzoic acid (1), phenylacetic acid (2), and 3-phenylpropionic acid (3).

FIGS. 4(a) and 4(b) depict illustrative embodiments of (A-A₀) versus mole ratio plots obtained from the absorption intensity titration of compound L with amino acids. FIG. 4(a) plots absorption intensity at the 214 nm band. FIG. 4(b) plots absorption intensity at the for 352 nm band. The symbols are: ▾=Asp, ▪=Glu, ▴=Trp, ▾=Tyr, =Phe, ▪=His, [symbol] =Cys, [symbol] =Gln, ▪=Lys, and ♦=Met.

FIGS. 5(a)-(d) depict illustrative embodiments computationally optimized, Becke 3-Parameter, Lee, Yang and Parr (B3LYP), structures for the complexes of compound L with, Trp (5(a)), Phe (5(b)), His (5(c)), and Tyr (5(d)).

FIG. 6 depicts an illustrative embodiment of a computed energy-minimized (using MM+ force field from Hyperchem) molecular structure of a complex containing 2 Phe molecules and 2 molecules of compound L.

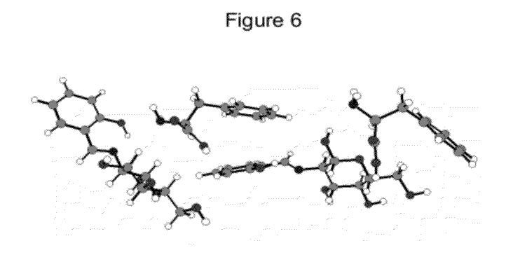

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. The present technology is also illustrated by the examples herein, which should not be construed as limiting in any way.

As used herein [A] refers to the concentration of an amino acid. “[L]” refers to the concentration of compound L.

“Alpha-amino acid” refers to an amino acid where the amino group is covalently bonded to the same carbon to which a carboxyl group is bonded.

“Aromatic alpha-amino acid” refers to an alpha-amino acid including a mono-, bi-, or tri-cyclic aryl or heteroaryl group. The aryl or heteroaryl group may be directly attached to the alpha-amino acid group or linked through another group (“linker group”) including by not limited to alkyl, alkenyl, or alkoxy group. In some embodiments the linker is a group with 1, 2, 3, 4, 5, or 6 carbon atoms. Examples of aromatic alpha-amino acids include, without limitation, histidine (His), phenylalanine (Phe), tryptophan (Trp), and Tyrosine (Tyr).

“Ester” refers to carboxyl groups in which the carboxylic hydrogen is replaced with a substituted or unsubstituted alkyl, cycloalkyl, cycloalkylalkyl, alkenyl, cycloalkenyl, cycloalkenylalkyl, aryl, aralkyl, heterocyclyl, heterocyclylalkyl, heteroaryl or heteroarylalkyl group. Thus, in an illustrative embodiment, an ester of an alpha-amino acid is one in which the carboxyl group alpha to the amino group is an ester and/or in which the carboxyl-containing side chain is an ester. The C-terminal amino acid residue of a peptide and/or amino acid side chain(s) in the peptide may also be an ester. Illustrative embodiments of esters include, but are not limited to, a methyl, ethyl or benzyl ester.

“Peptide” refers to a compound including at least two alpha amino acids in which each amino acid is attached to another via an amide bond between the carboxyl group of one amino acid and the alpha-amino group of the other amino acid.

“Control sample” refers to a sample against which the test sample is compared in order to assess the presence, absence and/or level of analyte in the test sample. As such, in the methods of the present technology, the control sample may include some or all of the constituents of the test sample, except for the analyte being assessed (negative control), e.g., except an aromatic alpha-amino acid, a peptide incorporating an aromatic alpha-amino acid, or a salt or ester thereof. Alternatively, the control sample may also include a known concentration of the analyte being assessed (positive control). Based on the present disclosure and the knowledge in the art, it is within the skill of one skilled in the art to select a proper control sample for performing the methods of the present technology. Depending on the detection method being used, the control sample can be a liquid sample or a solid sample. In some embodiments, the control sample is a liquid sample.

For the comparative measurement of sample fluorescence or absorption, the control sample may be dissolved in the same or substantially the same solvent/media as that of the test solvent. By “substantially the same solvent/media” is meant almost but not completely the same solvent/media.

“Test sample” refers to a sample which is to be tested for the presence and/or concentration of an analyte, such as an aromatic alpha-amino acid, a peptide incorporating an aromatic alpha-amino acid, or a salt or ester thereof. Depending on the detection method being used, the test sample can be a liquid sample or a solid sample. In some embodiments, the test sample is a liquid sample.

Compounds used and detected according to the present technology may include basic groups, such as amines and imines, and may therefore form salts with inorganic or organic acids. Such salts, include but are not limited to, salts of HClO₄, HCl, HBr, H₂SO₄, and H₃PO₄, as well as salts of acetic acid and trifluoroacetic acid. Other suitable salts include those described in P. Heinrich Stahl, Camille G. Wermuth (Eds.), Handbook of Pharmaceutical Salts: Properties, Selection, and Use, 2008, Wiley-VCH, Zurich (incorporated by reference in its entirety herein).

In general, “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like. In some embodiments, the substituted group bears 1-3 halogen, 1 or 2 hydroxyls, and or 1 or 2 alkoxy groups.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl and alkenyl groups as defined below.

Alkyl groups include straight chain and branched chain alkyl groups having from 1 to 12 carbon atoms, or, in some embodiments, from 1 to 10 carbons, 1 to 8, 1 to 6, or 1 to 4 carbon atoms. Examples of straight chain alkyl groups including but not limited to groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups.

Cycloalkyl groups include mono-, bi- or tricyclic alkyl groups having from 3 to 14 carbon atoms in the ring(s), or, in some embodiments, 3 to 12, 3 to 10, 3 to 8, or 3, 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl and the like.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group.

Alkenyl groups include straight and branched chain alkyl groups as defined above, except that at least one double bond exists between two carbon atoms. Thus, alkenyl groups have from 2 to 12 carbon atoms, and typically from 2 to 10 carbons or, in some embodiments, from 2 to 8, 2 to 6, or 2 to 4 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, butadienyl, pentadienyl, and hexadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group.

Aryl groups are cyclic aromatic hydrocarbons of 6-14 carbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl.

Heterocyclyl groups include non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass partially unsaturated and saturated ring systems, such as, for example, imidazolyl, imidazolinyl and imidazolidinyl groups. The phrase “heterocyclyl group” includes fused and bridged non-aromatic ring species including, for example, octohydro-[1H]-quinolizine and quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, oxiranyl, pyrrolidinyl, imidazolidinyl, pyrazolidinyl, thiazolidinyl, tetrahydrothiophenyl, tetrahydrofuranyl, pyrrolinyl, imidazolinyl, pyrazolinyl, thiazolinyl, piperidinyl, piperazinyl, morpholinyl, thiomorpholinyl, tetrahydropyranyl, tetrahydrothiopyranyl, oxathiane, dihydropyridyl, dihydrodithiinyl, dihydrodithionyl, homopiperazinyl, quinuclidinyl, and indolinyl groups.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl and benzofuranyl groups and include fused ring compounds in which only one of the rings is aromatic, such as 2,3-dihydro indolyl groups or 2,3-dihydrobenzofuranyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.”

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group.

In certain aspects, the present technology provides compounds of Formula I and complexes including a compound of Formula I:

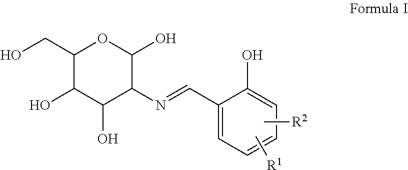

and an aromatic alpha-amino acid, a peptide incorporating an aromatic alpha-amino acid, or a salt or ester of any of the foregoing, wherein R¹ and R² are hydrogen or R¹ and R² together with the carbon atoms to which they are bonded form a phenyl ring.

In some embodiments of compounds of Formula I or complexes including such compounds, R¹ and R² are hydrogen. In other embodiments, the compound of Formula I is 1-(D-glucopyranosyl-2′-deoxy-2′-iminomethyl)-2-hydroxybenzene (or simply, compound L) of formula:

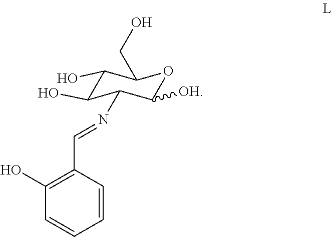

In some embodiments of complexes including compounds of Formula I, the aromatic alpha-amino acid or the peptide incorporating the aromatic alpha-amino acid has the Formula II:

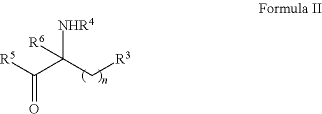

wherein
R³ is:

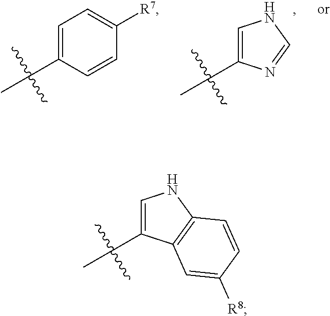

wherein R⁷ is hydrogen or hydroxyl;
R⁸ is hydrogen or hydroxyl;
R⁴ is hydrogen or an amino acyl moiety wherein the amino acyl moiety is an acyl moiety derived from an alpha-amino acid or a peptide;
R⁵ is hydroxyl or an amino moiety derived from an alpha-amino acid or a peptide;
R⁶ is hydrogen or methyl; and
n is 0 or 1.

In some embodiments in which the aromatic alpha-amino acid or the peptide incorporating the aromatic alpha-amino acid has the Formula II, R⁴ is hydrogen. In other embodiments, R⁵ is hydroxyl. In some embodiments, R⁴ is hydrogen and R⁵ is hydroxyl. In still other embodiments, R⁶ is hydrogen. In certain embodiments, n is 1. In some embodiments of complexes of the present technology, the aromatic alpha-amino acid is phenylalanine, tyrosine, histidine, or tryptophan.

Complexes including a compound of Formula I may include various amounts of an aromatic alpha-amino acid, a peptide incorporating an aromatic alpha-amino acid, salts and esters thereof. Thus, for example, the molar ratio of the compound of Formula I and the aromatic alpha-amino acid or the peptide, or a salt or ester thereof may range from about 1:1 to about 1:2. It will be understood that more than one complex may exist in the presence of others and that the concentration of any particular complex in a solution may vary depending on the relative amounts of the compound of Formula I and ligand(s) (i.e., aromatic alpha-amino acid(s), a peptide incorporating aromatic alpha-amino acid(s), salts and esters thereof) present. It is also to be understood that complexes of the present technology may exist in the presence of uncomplexed compounds of Formula I or uncomplexed aromatic alpha-amino acid, a peptide incorporating an aromatic alpha-amino acid, salts and esters thereof. In some embodiments of the present compounds and complexes, the compound of Formula I is compound L.

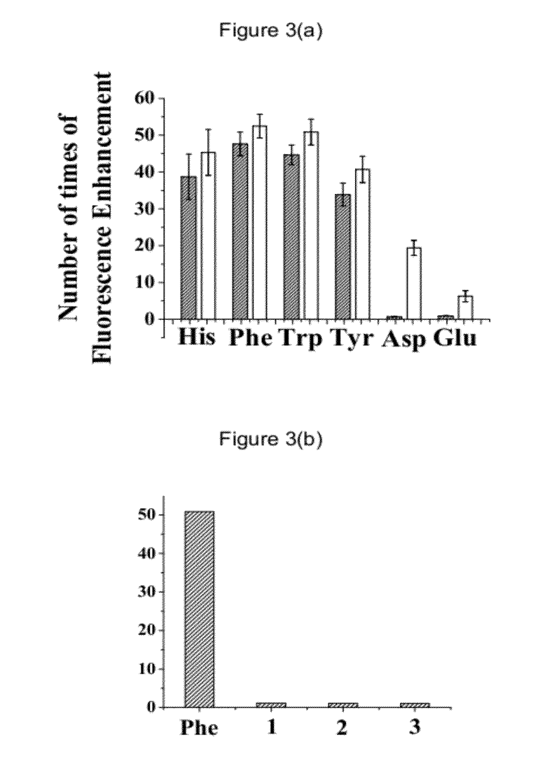

In some embodiments, the association constant (Kₐ) of the complex is from about 6000 M⁻¹ to about 30000 M⁻¹, from about 10000 M⁻¹ to about 25000 M⁻¹, and from about 15000 M⁻¹ to about 20000 M⁻¹. Complexes of compounds of Formula I with aromatic acids and non-aromatic alpha-amino acids are significantly weaker, showing the selectivity of compounds for Formula I in binding aromatic alpha-amino acids. For example, complexes of L with alanine and arginine exhibit Kₐ values of 3360±225 M⁻¹ and 1930±150 M⁻¹ respectively, and these are about one-sixth to one-tenth of that observed for His, Tyr, Trp, and Phe. Likewise, aromatic amino acids such as 3-phenyl propionic acid, phenylacetic acid and benzoic acid did not display any measurable binding as judged by fluorescent enhancement (see FIG. 3(b)). Kₐ values for the present complexes can be determined as described in the Examples, and by a variety of other methods, including NMR-based methods, that will be apparent to one of skill in the art upon reading this disclosure.

Compounds of Formula I may be synthesized by reacting 2-(D)-glucosamine or diastereomers or an enantiomer thereof with the corresponding aldehyde as schematically shown below:

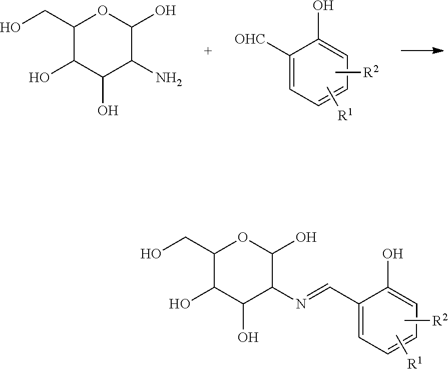

For example, the carbohydrate based receptor, compound L, was synthesized in one step by condensing glucosamine and salicylaldehyde in ethanol. (See also, Singhal, N. K. et al. Org. Lett. 2006, 8, 3525 and Villagran, M., et al. Boletin de la Sociedad Chilena de Quimica. 1994, 39, 121, each of which is incorporated herein by reference) Glucosamine is conveniently obtained by neutralizing the corresponding ammonium salt. A variety of bases, including organic bases (e.g., pyridine, triethylamine and the like) and inorganic bases (e.g., NaOH, KOH and the like) are useful for the neutralization.

In another aspect, the present technology provides methods of testing for the presence or absence of an aromatic alpha-amino acid, a peptide incorporating an aromatic alpha-amino acid, or a salt or ester thereof (e.g., any of the compounds of Formula II described herein). The methods include detecting the fluorescence emission intensity of a test sample including a compound of Formula I as shown above, and comparing the detected fluorescence emission intensity of the test sample to that of a control sample. A change in the fluorescence emission intensity of the test sample relative to the control sample indicates the presence of the aromatic alpha-amino acid, the peptide incorporating an aromatic alpha-amino acid, or the salt or ester thereof.

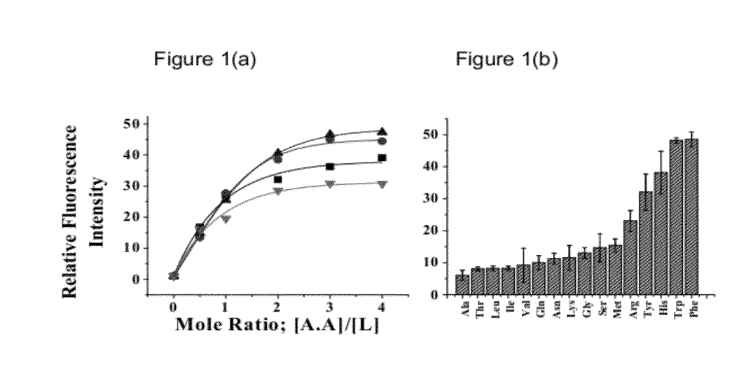

For example, using a negative control, an increase in the fluorescence emission intensity of the test sample relative to the control sample indicates the presence of the aromatic alpha-amino acid, the peptide incorporating an aromatic alpha-amino acid, or the salt or ester thereof. By comparison to non-aromatic amino acids, the intensity changes may, e.g., range from two to ten-fold (See FIG. 1(b)). In contrast, an unchanged (including little or no change) fluorescence emission intensity of the test sample relative to the negative control sample indicates the absence of detectable amounts of aromatic alpha-amino acid, the peptide incorporating an aromatic alpha-amino acid, or the salt or ester thereof in the test sample. In such assays, a “negative control” refers to a control sample which lacks the analyte (i.e., aromatic alpha-amino acid, the peptide incorporating an aromatic alpha-amino acid, or the salt or ester thereof). The negative control may optionally include the compound of Formula I. In an illustrative embodiment, the negative control sample contains substantially the same amount of the compound of Formula I as the test sample but lacks the aromatic alpha-amino acid, the peptide incorporating an aromatic alpha-amino acid, or the salt or ester thereof. By “substantially the same amount” is meant the same amount or nearly the same amount.

Alternately, where a positive control sample is used, a decrease in fluorescence intensity may indicate less of an aromatic amino acid than contained in the control sample, whereas an increase in such intensity may indicate a higher concentration of the aromatic alpha-amino acid than the control. In such assays, a “positive control” refers to a control sample which includes the analyte (as defined herein). The positive control may optionally include the compound of Formula I. In an illustrative embodiment, the positive control sample contains substantially the same amount of the compound of Formula I as the test sample and an equivalent amount of the analyte.

In the present methods, a variety of test samples may be assayed including but not limited to biological, food and environmental samples. A biological sample includes without limitation human and other mammalian body fluids such as serum, blood, or urine. In some embodiments, the test sample is an aqueous. The aqueous sample may include, without limitation, cell culture media, which may or may not be in contact with cells. Test samples may be taken from the body, food, or the environment, or may be prepared from the aforementioned sources by use of standard techniques such as digestion and extraction to isolate in whole or part, the aromatic alpha-amino acid or derivatives thereof (as disclosed herein) to be analyzed.

In some embodiments, the compound of Formula I may be added to the test sample as a solid or as a solution (e.g., as a solution in an organic solvent such as chloroform and/or acetonitrile and/or methanol, or as an aqueous organic solution such as an aqueous methanol or an aqueous acetonitrile solution). Alternatively, the test sample or an aliquot thereof may be added to the compound of Formula I or to a solution thereof. The test sample may also be prepared by adding the compound of Formula I or a solution thereof and an aliquot of the sample to be tested to a third solution. Various concentrations of compounds of Formula I may be used including but not limited to about 8 μM to about 200 μM. In other embodiments, the concentration of compounds of Formula I range from about 10 μM to about 100 μM, from about 25 μM to about 75 μM, or at about 50 μM. In another aspect, the present technology provides the compounds of Formula I, formulated for use in the methods described herein.

In some embodiments, the methods of the present technology can be used to detect the aromatic alpha-amino acid or derivatives thereof as disclosed herein (or simply, the aromatic alpha-amino acid or derivatives thereof) at a minimum concentration of about 1.5 ppm. In some embodiments, the concentration of the aromatic alpha-amino acid or derivatives thereof that can be detected in the test sample is at least about 2.0 ppm, at least about 3.0 ppm, at least about 4.0 ppm, or at least about 5.0 ppm. In some embodiments, the concentration of the aromatic alpha-amino acid or derivatives thereof that can be detected is in the range of about 1.5 ppm to about 500 ppm, about 2 ppm to about 400 ppm, about 4 ppm to about 300 ppm, about 5 ppm to about 100 ppm; or about 6 ppm to about 50 ppm.

In the methods of the present technology, the fluorescence of a compound of Formula I may be detected by essentially any suitable fluorescence detection device. Such devices typically include a light source for excitation of the fluorophore and a sensor for detecting emitted light. In addition, the fluorescence detection devices may contain a means for controlling the wavelength of the excitation light and a means for controlling the wavelength of the light detected by the sensor. Such means are referred to generically as filters and can include diffraction gratings, dichroic mirrors, or filters. Examples of suitable devices include fluorometers, spectrofluorometers and fluorescence microscopes. Many such devices are commercially available from companies such as Perkin-Elmer, Hitachi, Nikon, Molecular Dynamics, or Zeiss. In certain embodiments, the device is coupled to a signal amplifier and a computer for data processing.

Using the above devices, the fluorescence excitation and emission spectra of compounds of Formula I may be determined by standard techniques in the art. Thus, suitable excitation and emission wavelengths may be readily selected by those of skill in the art for the application at hand. Generally, compounds of Formula I may be excited at a wavelength ranging from about 220 nm to about 360 nm or from about 300 to about 40 and the emission monitored at a wavelength from about 330 nm to about 550 nm (so long as the emission wavelength is longer than the excitation wavelength). In certain embodiment, compound L may be excited at a wavelength of about 320 nm. In certain embodiments, the fluorescence emission intensity may be detected in the range from about 330 nm to about 500 nm.

In some embodiments, the methods of the present technology can be used to detect the aromatic alpha-amino acid or derivatives thereof in the presence of one or more other alpha-amino acids.

In another aspect, the present technology provides methods of testing for the presence or absence of tryptophan or a salt or ester thereof including: detecting the ultraviolet absorption intensity of a test sample including a compound of Formula I, and comparing the detected ultraviolet absorption intensity of the test sample to that of a control sample,
wherein a change (e.g., a decrease) in the ultraviolet absorption intensity of the test sample relative to the control sample indicates the presence of tryptophan or the salt or ester thereof in the test sample, and an unchanged (i.e., little or no change) ultraviolet absorption intensity of the test sample relative to the control sample indicates the absence of tryptophan or the salt or ester thereof in the test sample.

In some embodiments, the compound of Formula I is compound L. In other embodiments, the ultraviolet absorption intensity is detected at about 214 nm. In other embodiments, the test sample is an aqueous, biological, food or environmental sample. In some embodiments, the control sample contains substantially the same amount of the compound of Formula I as the test sample but lacks tryptophan or the salt or ester thereof.

### EXAMPLES

The present technology is further illustrated by the following examples, which should not be construed as limiting in any way. The following definitions are used herein.
AM1 Austin Model 1
d Doublet
DMSO Dimethylsulfoxide
ESI Electron spray ionization
FTIR Fourier transform infra-red
g Gram
HF Hartree Fock
MHz Megahertz
μL Microliter
mL Milliliter
μM Micromolar
mmol Millimole
MS Mass spectroscopy
m/z Mass/charge
nm Nanometer
NMR Nuclear magnetic resonance
ppm Parts per million
s Singlet
STO Slater type orbital
t Triplet

### Example 1

### Synthesis of Compound L

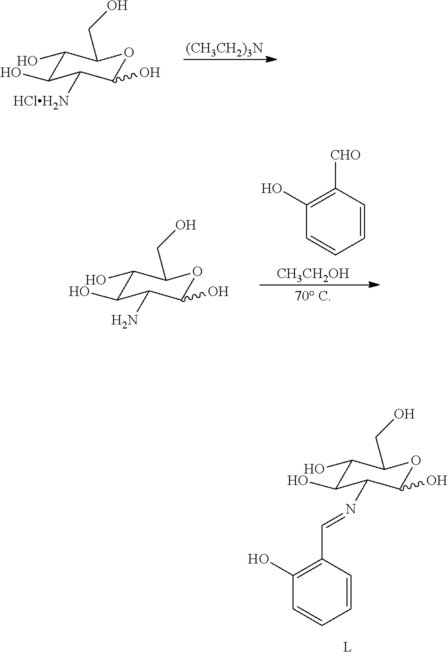

Glucosamine hydrochloride (0.215 g, 1 mmol) salt was neutralized with triethylamine in ethanol before was used in the synthesis. To this neutralized solution was added salicylaldehyde (0.15 ml, 1 mmol). The reaction mixture was refluxed for 6 hours at 60° C. The solid product formed, compound L, (0.25 gm) was filtered, washed with cold ethanol several times and diethyl ether, and dried under vacuum to provide compound L in 87% yield. ¹HNMR (DMSO-d₆, ppm): 3.25-3.80 (m, 5H, C2-H, C3-H, C4-H, C5-H), 4.54-4.95 (4d, 4H, C1-OH, C3-OH, C4-OH & C6-OH), 5.16-5.18 (d, H, C1-H, ³J_{C1-H-C2-H}, 5.5 Hz), 6.19-7.59 (2d, 2t, 4H, Ar—OH), 8.6 (S, H, CH═N), 13.2 (S, H, Ar—OH). ESI MS m/z=284 ([M+H]⁺, 100%).

### Example 2

### Fluorescence Titrations

Fluorescence emission spectra were determined using a Perkin-Elmer LS55 fluorescence spectrophotometer by exciting the samples at 320 nm and recording the emission spectra in the 330 nm-550 nm range. The bulk solutions of compound L and amino acids were prepared in methanol in which 400 μl (4%) of water was added to dissolve the amino acid. The bulk solution concentrations were maintained at 1×10⁻³ M. The measurements were made in 1 cm quartz cell and the effective concentration of L was maintained at 50 μM.

To demonstrate the sensitivity and selectivity of compound L for detecting amino acids, fluorescence titrations were performed using the twenty naturally occurring amino acids. Different mole ratios of each amino acid were added to the solution of compound L and the emission of all of the samples were measured after 24 h. Only the aromatic alpha-amino acids exhibited appreciable enhancement in the fluorescence emission intensity, which increased as a function of the mole ratio of amino acid added but saturated beyond two equivalents of amino acid (see FIG. 1(a)). All the other amino acids exhibited almost no or marginal changes in the fluorescence intensity (see FIG. 1(b)).

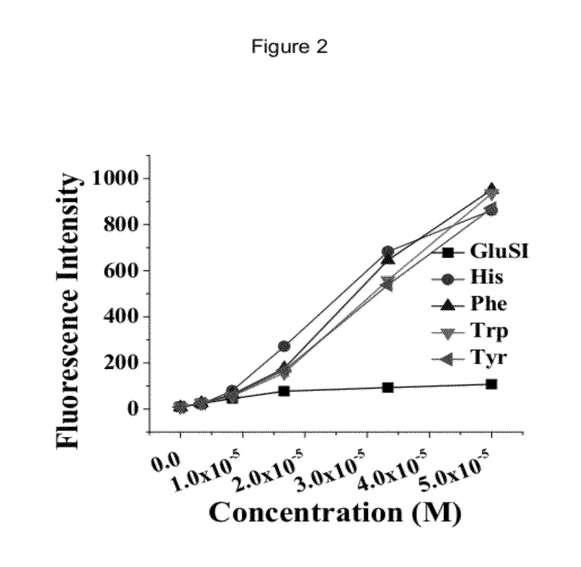

The association constants (Kₐ) for the complexes were derived from the fluorescence intensity changes by using Benesi-Hildebrand equation using the Origin Pro7.5 program. The Kₐ values were found to be about 19400±600 M⁻¹ for the complexes formed between aromatic alpha-amino acids and compound L. For two non-aromatic alpha-amino acids, alanine and arginine, Kₐ values of only about 3360±225 M⁻¹ (alanine) and about 1930±150 M⁻¹ (arginine) were obtained for the corresponding complexes with compound L. Based on these results, the aromatic alpha-amino acids demonstrate about 5 to about 10 times higher affinity, compared to the non-aromatic alpha-amino acids, in forming complexes with L. Based on concentration dependent fluorescence spectroscopy of compound L and the aromatic alpha-amino acid maintained in a 1:1 ratio, the lowest detection range for the detection of aromatic alpha-amino acids by compound L was found to be about 1.5 to about 3.0 ppm (FIG. 2).

The involvement of the —COOH group during the complexation of the amino acids with L was determined by fluorescence titrations employing amino acids, where the —COOH moiety of the amino acids were converted to the methyl ester (—COOCH₃). The results are shown in FIG. 3(a). For the amino acid esters, the fluorescence intensity enhancements were higher than those of the amino acids having free carboxylic moiety, suggesting the involvement of the carboxylic group in modulating the fluorescence intensity of compound L during the interaction. In case of Asp and Glu, where two such —COOH functions are present, there was a decrease in the fluorescence intensity of compound L when these two amino acids were added to it.

Without being bound by theory, the higher fluorescence intensity enhancement for aromatic alpha-amino acids may result from π-π interactions between the aromatic moiety of the amino acid and the aromatic moiety of a compound of Formula I. However presence of the aromatic moiety in the analyte may not be the only factor in the selective detection of the analyte by a compound of Formula I. To demonstrate this, titrations were carried out with aromatic carboxylic acids, 3-phenyl propionic acid, phenylacetic acid and benzoic acid. No significant enhancement of the fluorescence intensity of compound L was observed upon the addition of these molecules as shown in FIG. 3(b). The result demonstrated that L may recognize aromatic alpha-amino acids but not the corresponding aromatic carboxylic acids and further demonstrated the selectivity of L's identification of aromatic alpha-amino acids.

### Example 3

### Absorption Measurements

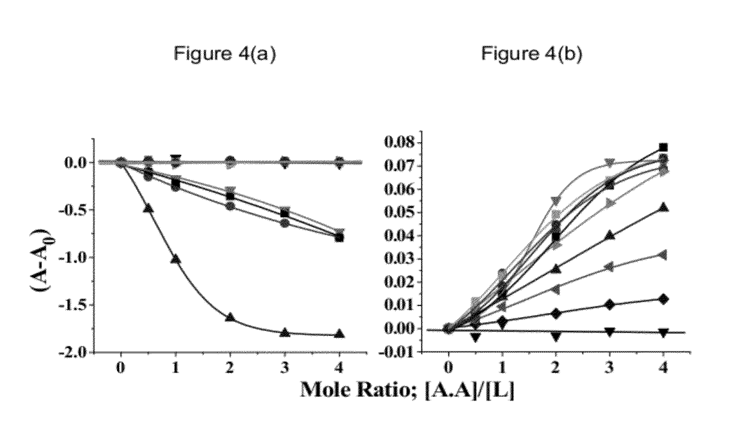

To further support the binding of amino acids to L, absorption titrations were carried out. The amino acids Asp and Glu, which exhibited almost no change in the fluorescence intensity when added to L (FIG. 3(a)), also exhibited no change in the absorption spectra of compound L (data not shown). In all other cases, (FIG. 4) a new band appeared at 352 nm indicating the formation of a complex between compound L and the amino acid.

For the aromatic alpha-amino acids, in addition to the appearance of this new band at 352 nm, the absorbance of 214 nm band diminished as a function of the concentration of added aromatic alpha-amino acid (data not shown), which was most prominent for Trp and followed the order: Trp>>Phe, His, Tyr. No change in the absorbance was observed in the 214 nm band with other amino acids (FIG. 4(a)), supporting the involvement of π-π interaction between compound L and the aromatic side chain of the aromatic alpha-amino acids. This observation is consistent with the report that absorption spectral changes may support the existence of π-π interactions between aromatic alpha-amino acids and another aromatic moiety. See, e.g., Jugun, P. C., Acharya, A., Kumar, A. and Rao, C. P. J. Phys. Chem. B 2009, 113, 12075, incorporated herein by reference. The reversibility of the present glycoconjugate chemosensor ensemble has been demonstrated by titrating the system with ethidium bromide, which is a well known DNA intercalator, reverses the conjugated species formed by quenching the fluorescence intensity (data not shown).

### Example 5

### Mass Spectroscopy

The stoichiometry of the species formed between L and Phe or Trp were determined by MALDI-TOF mass spectra by observing the peaks at m/z=473 and 472 respectively. These peaks correspond to the formation of (L+Phe+Na) and (L+Trp−H₂O+3H⁺) species.

### Example 6

### Computational Determination of Complex Structures and Complexation Energetics

Computational studies were carried out employing semi empirical, ab initio, and finally density functional theory (DFT) calculation, in a cascade fashion. As the formation of a 1:1 complex between L and the aromatic amino acids was supported by MALDI-TOF-MS and the Benesi-Hildebrand plot (data not shown), the 1:1 species were optimized using Gaussian 03 package, Frisch, M. J. et al., obtained from Gaussian, Inc., Wallingford Conn., (Gaussian 03, Revision C.02) 2004.

Prior to assuming the initial guess model for computational calculations, compound L and the amino acids, Phe, Trp, His, Tyr, were independently optimized by using different theories in the cascade fashion discussed above. The corresponding complexes of compound L with these amino acids were made by simply placing the amino acid far away from compound L in such a way that the side chain of amino acid is pointed towards the salicyl moiety of compound L. Then the complexes were also optimized in a cascade fashion by going through AM1→HF/STO-3G→HF/3-21G→HF/6-31G→B3LYP/3-21G→B3LYP/6-31G.

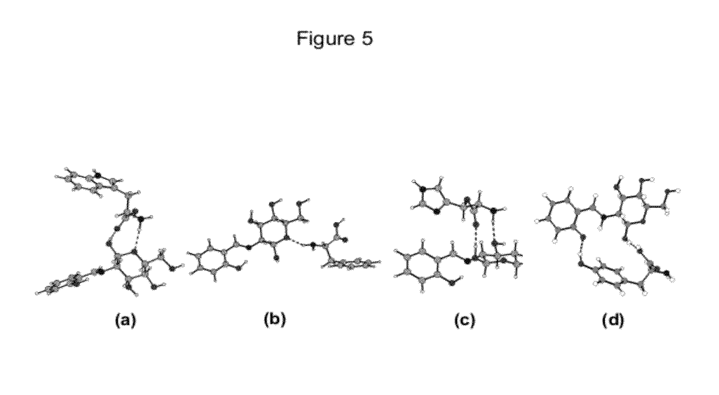

At the B3LYP/6-31G level, the complexes of compound L with Trp, Phe, and His involved the interaction of the carboxylic and amine moiety of the amino acid with compound L. The complex of compound L with Trp was stabilized by two hydrogen bond interactions (FIG. 5(a)) formed between the carbonyl and amine functional groups of Trp with the C1-OH and the pyranosyl ring oxygen of compound L, forming a 9-atom ring (C═O . . . H—OC1 & HNH . . . O_{pyranose}). In the complex of compound L with Phe, there was one HNH . . . O_{pyranose} hydrogen bond present (FIG. 5(b)). In the His complex of compound L, the —C═O and the —NH₂ groups of His formed hydrogen bonds with the C1-OH and the C3-OH (HNH . . . OHC3 & C═O . . . HOC1) through the formation of an 11-atom ring (FIG. 5(c)). The complex of Tyr with L was stabilized by two O—H . . . O hydrogen bond interactions (FIG. 5(d)).

At B3LYP/6-31G level, the stabilization energies were computed using the formula ΔEₛ=E_{c}−[E_{L}+Eₐₐ], where E_{c} is the total energy of the complex, E_{L} is the total energy of the glycoconjugate, and Eₐₐ is the total energy of the amino acid, yielded −3.9 kcal/mol, −10.3 kcal/mol, −7.9 kcal/mol and −17.3 kcal/mol respectively for the Phe, Trp, His and Tyr complexes and these are commensurate with the H-bond interactions present in these complexes.

### Equivalents

The present disclosure is not to be limited in terms of the particular aspects and embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular aspects and embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A complex comprising a compound of Formula I: 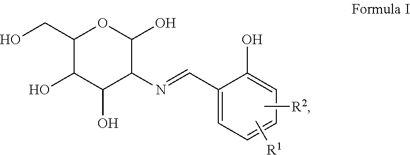
and an aromatic alpha-amino acid, a peptide incorporating an aromatic alpha-amino acid, or a salt or ester of any of the foregoing, wherein
R¹ and R² are hydrogen or R¹ and R² together with the carbon atoms to which they are bonded form a phenyl ring.

2. The complex of claim 1, wherein R¹ and R² are hydrogen.

3. The complex of claim 1, wherein the compound of Formula I is 1-(D-glucopyranosyl-2′-deoxy-2′-iminomethyl)-2-hydroxybenzene.

4. The complex of claim 1, wherein the aromatic alpha-amino acid, the peptide incorporating an aromatic alpha-amino acid, or an ester thereof has the Formula II: 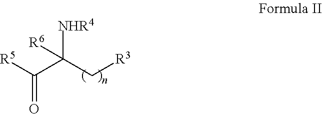
wherein
R³ is: 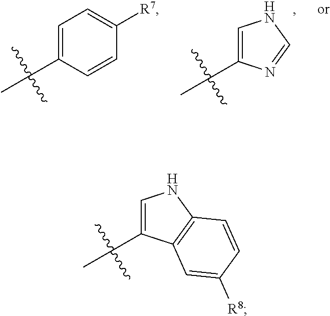
wherein R⁷ is hydrogen or hydroxyl;
R⁸ is hydrogen or hydroxyl;
R⁴ is hydrogen or an amino acyl moiety wherein the amino acyl moiety is an acyl moiety derived from an alpha-amino acid or a peptide;
R⁵ is hydroxyl or an amino moiety derived from an alpha-amino acid or a peptide;
R⁶ is hydrogen or methyl; and
n is 0 or 1.

5. The complex of claim 4, wherein R⁴ is hydrogen and/or R⁵ is hydroxyl.

6. The complex of claim 4, wherein R⁶ is hydrogen and/or n is 1.

7. The complex of claim 1 wherein the aromatic alpha-amino acid is phenylalanine, tyrosine, histidine, or tryptophan.

8. The complex of claim 7 wherein the compound of Formula I is 1-(D-glucopyranosyl-2′-deoxy-2′-iminomethyl)-2-hydroxybenzene.

9. A method of testing for the presence an aromatic alpha-amino acid, a peptide incorporating an aromatic alpha-amino acid, or a salt or ester of any of the foregoing comprising:
detecting the fluorescence emission intensity of a test sample comprising a compound of Formula I: 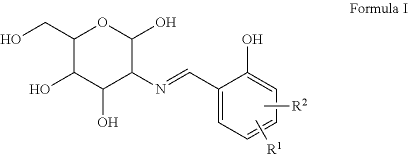
wherein
R¹ and R² are hydrogen or R¹ and R² together with the carbon atoms to which they are bonded form a phenyl ring;
and
comparing the detected fluorescence emission intensity of the test sample to that of a control sample,
wherein a change in the fluorescence emission intensity of the test sample relative to the control sample indicates the presence of the aromatic alpha-amino acid, the peptide incorporating an aromatic alpha-amino acid, or the salt or ester of any of the foregoing.

10. The method of claim 9, wherein the compound of Formula I is 1-(D-glucopyranosyl-2′-deoxy-2′-iminomethyl)-2-hydroxybenzene.

11. The method of claim 9, wherein the aromatic alpha-amino acid, the peptide incorporating an aromatic alpha-amino acid, or an ester thereof has the Formula II: 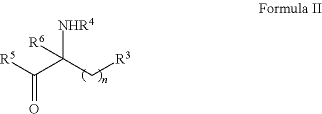
wherein
R³ is: 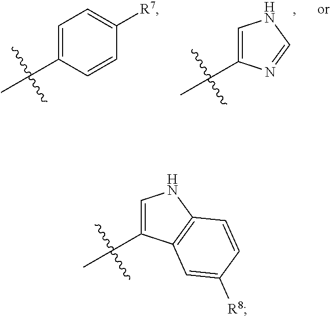
R⁷ is hydrogen or hydroxyl;
R⁸ is hydrogen or hydroxyl;
R⁴ is hydrogen or an amino acyl moiety wherein the amino acyl moiety is an acyl moiety derived from an alpha-amino acid or a peptide incorporating alpha-amino acids;
R⁵ is hydroxyl or an amino moiety derived from an alpha-amino acid or a peptide incorporating alpha-amino acids;
R⁶ is hydrogen or methyl; and
n is 0 or 1.

12. The method of claim 11, wherein R⁴ is hydrogen and/or R⁵ is hydroxyl.

13. The method of claim 11, wherein R⁶ is hydrogen and/or n is 1.

14. The method of claim 11, wherein the aromatic alpha-amino acid is phenylalanine, tyrosine, histidine, tryptophan or a mixture of any two or more thereof.

15. The method of claim 14, wherein the compound of Formula I is 1-(D-glucopyranosyl-2′-deoxy-2′-iminomethyl)-2-hydroxybenzene.

16. The method of claim 9, wherein the fluorescence emission intensity is detected in the range from about 330 nm to about 500 nm.

17. The method of claim 9, wherein the control sample contains substantially the same amount of the compound of Formula I as the test sample but lacks the aromatic alpha-amino acid, the peptide incorporating an aromatic alpha-amino acid, or the salt or ester thereof.

18. A method of testing for the presence or absence of tryptophan or a salt thereof comprising:
detecting the ultraviolet absorption intensity of a test sample comprising a compound of Formula I: 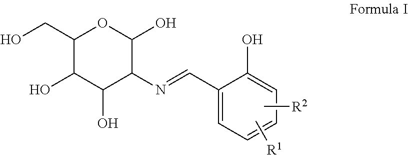
wherein
R¹ and R² are hydrogen or R¹ and R² together with the carbon atoms to which they are bonded form a phenyl ring;
and
comparing the detected ultraviolet absorption intensity of the test sample to that of a control sample,
wherein a decrease in the ultraviolet absorption intensity of the test sample relative to the control sample indicates the presence of tryptophan in the test sample, and an unchanged ultraviolet absorption intensity of the test sample relative to the control sample indicates the absence of tryptophan in the test sample.

19. The method of claim 18, wherein the compound of Formula I is 1-(D-glucopyranosyl-2′-deoxy-2′-iminomethyl)-2-hydroxybenzene.

20. The method of claim 18, wherein the ultraviolet absorption intensity is detected at about 214 nm.
